(19) United States
(12) Patent Application Publication
(10) Pub. No.: US 2024/0237804 A1
(43) Pub. Date: Jul. 18, 2024

(54) PHOTOCHROMIC ARTIFICIAL NAIL COMPOSITIONS AND METHODS

(71) Applicant: Taquilla KENNEDY, Riverside, CA (US)

(72) Inventor: Taquilla KENNEDY, Riverside, CA (US)

(21) Appl. No.: 18/097,186

(22) Filed: Jan. 13, 2023

(51) Int. Cl. A45D 31/00; A45D 29/00

(52) U.S. Cl. CPC A45D 31/00; A45D 29/00; A45D 2200/25

(57) ABSTRACT

Provided herein are compositions, kits, and methods useful for preparation of artificial nails, such as acrylic nails or dip powder nails, with a photochromic color agent that changes color reversibly upon exposure to light.

### FIELD

Provided herein are compositions, kits, and methods useful for preparation of artificial nails, such as acrylic nails or dip powder nails, with a photochromic color agent that changes color reversibly upon exposure to light.

### BACKGROUND OF THE INVENTION

Artificial nails have been used for centuries to decorate and enhance the beauty of human hands. Although early artificial nails used natural materials, modern methods of preparing artificial nails use polymers, such as polyacrylate, as the nail material. These new materials allow the incorporation of new color or contrast elements.

Therefore, there is a continuing need for compositions, kits, and methods useful for preparation of artificial nails with novel, attractive features that enhance their esthetic appeal or their other properties.

### SUMMARY OF THE INVENTION

Provided herein are compositions, kits, and methods useful for preparation of artificial nails (e.g., acrylic nails; dip powder nails) with photochromic color agents. In certain embodiments, the photochromic color agent changes color reversibly upon exposure to light (e.g., ultraviolet light; sunlight).

In certain embodiments, provided herein are artificial nail precursor compositions, the composition including:
a polyacrylate precursor; and
a photochromic color agent; wherein the photochromic color agent changes color reversibly upon exposure to ultraviolent light.

In certain embodiments, provided herein is the artificial nail precursor composition as otherwise disclosed, wherein the polyacrylate precursor comprises poly(methyl methacrylate) (PMMA). In certain embodiments, the polyacrylate precursor comprises 2-cyanoacrylate.

In certain embodiments, provided herein is the artificial nail precursor composition as otherwise disclosed, wherein upon exposure to ultraviolet light, the photochromic color agent changes from white to purple, from blue to purple, from orange to magenta, from yellow to orange, from yellow to magenta, from yellow to teal, from yellow to purple, or from white to pink.

In certain embodiments, provided herein is the artificial nail precursor composition as otherwise disclosed, further comprising a photochromic glitter.

In certain embodiments, provided herein are kits for preparation of an artificial nail, the kit comprising:
the artificial nail precursor composition as otherwise disclosed herein; and
instructions for preparation of the artificial nail.

In certain embodiments, provided herein are kits for preparation of the artificial nail as otherwise disclosed herein, further comprising an acrylic monomer; wherein the acrylic monomer comprises ethyl methacrylate.

In certain embodiments, provided herein are kits for preparation of the artificial nail as otherwise disclosed herein, further comprising a photochromic glitter.

In certain embodiments, provided herein are methods for the preparation of an acrylic nail, the methods comprising:
combining the artificial nail precursor as otherwise disclosed herein with an acrylic nail monomer to form a nail precursor mixture; and
shaping the nail precursor mixture to a nail shape.

In certain embodiments, provided herein are methods for the preparation of an acrylic nail, further comprising applying glitter to an outer surface of the nail shape.

In certain embodiments, provided herein are acrylic nails prepared by a process as otherwise disclosed herein.

In certain embodiments, provided herein are methods for the preparation of a dip powder nail, the methods comprising:
combining contacting a base-gel-coated nail with a dip powder comprising the artificial nail precursor as otherwise disclosed herein to form a dip-powder-coated nail; and
contacting the dip-powder-coated nail with an activator to form the dip powder nail.

In certain embodiments, provided herein are methods for the preparation of a dip powder nail, further comprising applying glitter to an outer surface of the dip-powder-coated nail or the dip powder nail.

In certain embodiments, provided herein are dip powder nails prepared by a process as otherwise disclosed herein.

### DETAILED DESCRIPTION

### Description of Exemplary Embodiments

Provided herein are compositions, kits, and methods useful for preparation of artificial nails including photochromic color agents. In certain embodiments, the photochromic color agent changes color reversibly upon exposure to light (e.g., ultraviolet light; sunlight).

### Definitions

When referring to the compounds provided herein, the following terms have the following meanings unless indicated otherwise. Unless defined otherwise, all technical and scientific terms used herein have the same meaning as is commonly understood by one of ordinary skill in the art. In case of a plurality of definitions for a term herein, those in this section prevail unless stated otherwise.

All publications, patent applications, patents, and other references mentioned herein are incorporated herein by reference in their entirety. In case of conflict, the present application will control.

The articles “a,” “an,” and “the” as used herein not only include certain embodiments with a single member, but also may include embodiments with more than one member. For example, an aspect “comprising a photochromic color agent and a photochromic glitter” should be understood as presenting certain embodiments with at least a second photochromic color agent, at least a second photochromic glitter, or both.

Similarly, the term “or” as used herein is a Boolean “or,” unless the alternatives cannot be combined without logical incompatibility. For example, an aspect “comprising a photochromic agent selected from A, B, or C” should be understood as applying to embodiments comprising A and B; B and C; A and C; or A, B, and C.

The term “about” as used herein to modify a numerical value indicates a defined range around that value. If “X” were the value, “about X” would generally indicate a value from 0.95X to 1.05X. Any reference to “about X” specifically indicates at least the values X, 0.95X, 0.96X, 0.97X, 0.98X, 0.99X, 1.01X, 1.02X, 1.03X, 1.04X, and 1.05X. Thus, “about X” is intended to teach and to provide written description support for a claim limitation of, e.g., “0.98X.” When “about” is applied to the beginning of a numerical range, it applies to both ends of the range. Thus, “from about 5 to 20%” is equivalent to “from about 5% to about 20%.” When “about” is applied to the first value of a set of values, it applies to all values in that set. Thus, “about 7, 9, or 11%” is equivalent to “about 7%, about 9%, or about 11%.”

The term “color agent” as used herein, and unless otherwise specified, refers to a compound or mixture of compounds added to a composition to impart a particular color to the composition. In certain preferred embodiments, one or more color agents is photochromic (i.e., a photochromic agent).

The term “cuticle” as used herein, and unless otherwise specified, refers to the thin layer of dead skin proximate to the eponychium on the nail bed.

The term “eponychium” as used herein, and unless otherwise specified, refers to the living tissue at the base of the nail bed closer to the palm.

The term “nail plate” as used herein, and unless otherwise specified, refers to a hard, exposed portion of the flat, unliving, keratin-rich tissue at the end of the fingers and toes. The nail plate or a portion thereof is the site of attachment for the artificial nail.

The term “photochromic” as used herein, and unless otherwise specified, refers to a property of changing color upon exposure to light (e.g., daylight; ultraviolet light).

The term “polyacrylate” as used herein, and unless otherwise specified, refers to a polymer of an R¹(CO)CR²═CR³(R⁴) monomer (i.e., an acrylate) or (NC)CR²═CR³(R⁴) monomer (i.e., a cyanoacrylate). In a preferred embodiment, R¹ is C₁₋₆alkyl or C₁₋₆alkyoxy, and R², R³, and R⁴ independently selected from the group including H and C₁₋₆alkyl (e.g., methyl). In certain embodiments, a polyacrylate is prepared from a monomer or mixture of monomers selected from ethyl acrylate, methyl acrylate, ethyl methacrylate, methyl methacrylate, or 2-cyanoacrylate. In certain embodiments, a polyacrylate is prepared from a derivative of acrylic acid (e.g., an acrylate ester) or mixture of such derivatives.

All percentage compositions are by weight (i.e., w/w) unless otherwise noted.

### Compositions

In certain embodiments, provided herein are artificial nail precursor compositions, the composition including:
a polyacrylate precursor; and
a photochromic color agent; wherein the photochromic color agent changes color reversibly upon exposure to ultraviolent light.

In certain embodiments, provided herein is the artificial nail precursor composition as otherwise disclosed, wherein the polyacrylate precursor comprises poly(methyl methacrylate) (PMMA). In certain embodiments, the polyacrylate precursor comprises 2-cyanoacrylate.

In certain embodiments, the artificial nail precursor composition includes from about 95% to 35% (w/w) of the polyacrylate precursor (e.g., about 99, 98, 97, 96, 95, 94, 93, 92, 91, 90, 89, 88, 87, 86, 85, 80, 75, 70, 65, 60, 55, 50, 45, 40, 35, or 30%). In certain embodiments, the artificial nail precursor composition includes from about 85% to 40% of the polyacrylate precursor (e.g., about 85, 84, 83, 82, 81, 80, 79, 78, 77, 76, 75, 70, 65, 60, 55, 50, 45, 40, or 35%). In certain embodiments, the artificial nail precursor composition includes from about 75% to 40% of the polyacrylate precursor (e.g., about 75, 74, 73, 72, 71, 70, 69, 68, 67, 66, 65, 60, 55, 50, 45, 40, or 35%). In certain embodiments, the artificial nail precursor composition includes from about 65% to 40% of the polyacrylate precursor (e.g., about 65, 64, 63, 62, 61, 60, 59, 58, 57, 56, 55, 54, 53, 52, 51, 50, 49, 48, 47, 46, 45, 44, 43, 42, 41, or 40%). In certain embodiments, the artificial nail precursor composition includes about 50% of the polyacrylate precursor.

In certain embodiments, the artificial nail precursor composition includes from about 5% to 65% (w/w) of the photochromic agent (e.g., about 1, 2, 3, 4, 5, 6, 7, 8, 9, 10, 15, 20, 25, 30, 35, 40, 45, 50, 55, 60, or 65%). In certain embodiments, the artificial nail precursor composition includes from about 35% to 60% (w/w) of the photochromic agent (e.g., about 30, 35, 40, 45, 50, 55, 56, 57, 58, 59, or 60%). In certain embodiments, the artificial nail precursor composition includes from about 40% to 55% (w/w) of the photochromic agent (e.g., about 40, 41, 42, 43, 44, 45, 46, 47, 48, 49, 50, 51, 52, 53, 54, or 55%).

In certain embodiments, provided herein is the artificial nail precursor composition as otherwise disclosed, wherein upon exposure to ultraviolet light, the photochromic color agent changes from white to purple, from blue to purple, from orange to magenta, from yellow to orange, from yellow to magenta, from yellow to teal, from yellow to purple, or from white to pink. In certain embodiments, the photochromic color agent changes from white to purple. In certain embodiments, the photochromic color agent changes from blue to purple. In certain embodiments, the photochromic color agent changes from orange to magenta. In certain embodiments, the photochromic color agent changes from yellow to orange. In certain embodiments, the photochromic color agent changes from yellow to magenta. In certain embodiments, the photochromic color agent changes from yellow to teal. In certain embodiments, the photochromic color agent changes from yellow to purple. In certain embodiments, the photochromic color agent changes from white to pink.

In certain embodiments, provided herein is the artificial nail precursor composition as otherwise disclosed, further comprising a glitter (e.g., a photochromic glitter). In certain embodiments, the photochromic glitter includes a second photochromic color agent as otherwise disclosed herein.

### Kits

In certain embodiments, provided herein are kits for preparation of an artificial nail, the kit comprising:
an artificial nail precursor composition as otherwise disclosed herein; and
instructions for preparation of the artificial nail.

In certain embodiments, provided herein are kits for preparation of the artificial nail as otherwise disclosed herein, further comprising an acrylic monomer; wherein the acrylic monomer comprises ethyl methacrylate.

In certain embodiments, provided herein are kits for preparation of the artificial nail as otherwise disclosed herein, further comprising a glitter (e.g., a photochromic glitter).

The kits can include a compound or composition provided herein and instructions providing information to a user for preparing and applying the artificial nail. Instructions may be provided in printed form or in the form of an electronic medium such as a floppy disc, CD, or DVD, or in the form of a website address where such instructions may be obtained.

In some embodiments, suitable packaging is provided. As used herein, “packaging” includes a solid matrix or material customarily used in a system and capable of holding within fixed limits a composition provided herein. Such materials include glass and plastic (e.g., polyethylene, polypropylene, and polycarbonate) bottles, vials, paper, plastic, and plastic-foil laminated envelopes and the like.

### Methods

In certain embodiments, provided herein are methods for the preparation of an acrylic nail, the methods comprising:
combining the artificial nail precursor as otherwise disclosed herein with an acrylic nail monomer to form a nail precursor mixture; and
shaping the nail precursor mixture to a nail shape.

In certain embodiments, provided herein are methods for the preparation of an acrylic nail, further comprising applying glitter (e.g., photochromic glitter) to an outer surface of the nail shape.

In certain embodiments, provided herein are acrylic nails prepared by a process as otherwise disclosed herein.

In certain embodiments, provided herein are methods for the preparation of a dip powder nail, the methods comprising:
combining contacting a base-gel-coated nail with a dip powder comprising the artificial nail precursor as otherwise disclosed herein to form a dip-powder-coated nail; and
contacting the dip-powder-coated nail with an activator to form the dip powder nail.

In certain embodiments, provided herein are methods for the preparation of a dip powder nail, further comprising applying glitter to an outer surface of the dip-powder-coated nail or the dip powder nail.

In certain embodiments, provided herein are dip powder nails prepared by a process as otherwise disclosed herein.

### Preparation of Compositions

The compositions provided herein can be prepared, isolated or obtained by any method apparent to those of skill in the art (e.g., by mixing solid powders). They can be packaged together, or packaged separately, for the convenience of the practitioner of skill in the art. Exemplary methods of preparation are described in detail in the examples below.

### EXAMPLES

As used herein, the symbols and conventions used in these processes, schemes and examples, regardless of whether a particular abbreviation is specifically defined, are consistent with those used in the contemporary technical literature.

For the following examples, standard application methods known to those skilled in the art can be used. All treatments are conducted at room temperature unless otherwise noted.

### Example 1

### Preparation of Photochromic Nail Powder

To a container is added ethyl methacylate powder (1 part) and a photochromic agent (1 part) that turns from white to purple upon exposure to ultraviolet light. The container is closed, and the components are mixed by shaking until they are approximately homogeneous.

### Example 2

### Application of Acrylic Nail

A nail is cleaned, trimmed, and gently buffed. If necessary to provide a better surface, the cuticles are removed and the eponychium is gently pushed back. The nail surface is treated with nail primer, which is allowed to dry. A drop of glue is applied to an artificial nail. The artificial nail is then carefully applied to the buffed nail without touching the glue to the epinychium or the tissue around the nail.

The liquid activator and the photochromic nail powder of Example 1 are placed in separate containers and combined (e.g., with a brush) to prepare a small, moist ball of artificial nail precursor material. This artificial nail precursor material is applied onto the nail tip with a brush to form the nail, with care taken not to paint the precursor material onto the flesh around the nail. The new acrylic nail is allowed to dry (i.e., to cure). Optionally, glitter or a coat of polish (e.g., a clear, protective coat) is applied to the acrylic nail after curing.

### Example 3

### Application of Dip Powder Nail

A clean nail is painted with a thin, even layer of a commercially available base gel. If necessary to provide a better surface, the cuticles are gently pushed back. Before the base gel dries, the nail is treated with the photochromic nail powder of Example 1, either by dipping the nail into the nail powder or by pouring the nail powder onto the nail. Any excess powder on the nail was removed by tapping. The dip or glitter is gently pushed down or off the surrounding surfaces if needed.

A dip powder activator is then painted on the dip-powder-coated nail to cure the artificial nail. At least one coat of the activator is applied, then allowed to cure before application of another coat of activator, glitter, or a coat of polish (e.g., a top or protective coat).

All publications and patent, applications cited in this specification are herein incorporated by reference as if each individual publication or patent application were specifically and individually indicated to be incorporated by reference. While the claimed subject matter has been described in terms of various embodiments, the skilled artisan will appreciate that various modifications, substitutions, omissions, and changes may be made without departing from the spirit thereof. Accordingly, it is intended that the scope of the subject matter limited solely by the scope of the following claims, including equivalents thereof.

1. An artificial nail precursor composition, the composition comprising:
a polyacrylate precursor; and
a photochromic color agent; wherein the photochromic color agent changes color reversibly upon exposure to ultraviolent light.

2. The artificial nail precursor composition of claim 1, wherein the polyacrylate precursor comprises poly(methyl methacrylate) (PMMA).

3. The artificial nail composition of claim 1, wherein the polyacrylate precursor comprises 2-cyanoacrylate.

4. The artificial nail precursor composition of claim 1, wherein upon exposure to ultraviolet light, the photochromic color agent changes from white to purple, from blue to purple, from orange to magenta, from yellow to orange, from yellow to magenta, from yellow to teal, from yellow to purple, or from white to pink.

5. The artificial nail precursor composition of claim 1, further comprising a photochromic glitter.

6. A kit for preparation of an artificial nail, the kit comprising:
the artificial nail precursor composition of claim 1; and
instructions for preparation of the artificial nail.

7. The kit of claim 6, further comprising an acrylic monomer;
wherein the acrylic monomer comprises ethyl methacrylate.

8. The kit of claim 6, further comprising a photochromic glitter.

9. A method for the preparation of an acrylic nail, the method comprising:
combining the artificial nail precursor of claim 1 with an acrylic nail monomer to form a nail precursor mixture; and
shaping the nail precursor mixture to a nail shape.

10. The method of claim 9, wherein the method further comprises applying glitter to an outer surface of the nail shape.

11. The acrylic nail prepared by the process of claim 9.

12. A method for the preparation of a dip powder nail, the method comprising:
contacting a base-gel-coated nail with a dip powder comprising the artificial nail precursor of claim 1 to form a dip-powder-coated nail; and
contacting the dip-powder-coated nail with an activator to form the dip powder nail.

13. The method of claim 12, wherein the method further comprises applying glitter to an outer surface of the dip-powder-coated nail or the dip powder nail.

14. The dip powder nail prepared by the process of claim 12.
